SEQUENCE LISTING (23 sequences)

SEQ ID NO: 1 - protein, 15 aa, Artificial Sequence
  Description of Artificial Sequence Synthetic peptide
MDEPSPLAQPLELNQ

SEQ ID NO: 2 - protein, 15 aa, Artificial Sequence
  Description of Artificial Sequence Synthetic peptide
AEVIRILSTMDSPST

SEQ ID NO: 3 - DNA, 28 bp, Artificial Sequence
  Description of Artificial Sequence PCR primer
gcgaattcacatcttcagaagaaagagc

SEQ ID NO: 4 - DNA, 29 bp, Artificial Sequence
  Description of Artificial Sequence PCR primer
gcgtcgacttaatcattggagtcttgtgg

SEQ ID NO: 5 - DNA, 28 bp, Artificial Sequence
  Description of Artificial Sequence PCR primer
gcgaattcacatcttcagaagaaagagc

SEQ ID NO: 6 - DNA, 30 bp, Artificial Sequence
  Description of Artificial Sequence PCR primer
gcgtcgactcagcccttgaagagcttttcc

SEQ ID NO: 7 - DNA, 27 bp, Artificial Sequence
  Description of Artificial Sequence PCR primer
gcgaattccgggaaaagctcttcaagg

SEQ ID NO: 8 - DNA, 29 bp, Artificial Sequence
  Description of Artificial Sequence PCR primer
gcgtcgacttaatcattggagtcttgtgg

SEQ ID NO: 9 - DNA, 26 bp, Artificial Sequence
  Description of Artificial Sequence PCR primer
gcgaattcgaaggaaccccatacctg

SEQ ID NO: 10 - DNA, 29 bp, Artificial Sequence
  Description of Artificial Sequence PCR primer
gcgtcgacttaatcattggagtcttgtgg

SEQ ID NO: 11 - DNA, 27 bp, Artificial Sequence
  Description of Artificial Sequence PCR primer
gcggatccacagcttcaacagaaaggg

SEQ ID NO: 12 - DNA, 26 bp, Artificial Sequence
  Description of Artificial Sequence PCR primer
gcgtcgactcagtagtggctgtgggg

SEQ ID NO: 13 - DNA, 5702 bp, Mus musculus
gtcctcggcgcttggaagtacggatcttttttctcggagaaaagttcactggaactggaagaaatggatttatctgccgtccaaattcaagaagtacaaaatgtccttcatgctatgcagaaaatcttagagtgtccgatctgtttggaactgatcaaagaacctgtttccacaaagtgtgaccacatattttgcaaattttgtatgctgaaacttcttaaccagaagaaagggccttcacaatgtcctttgtgtaagaatgagataaccaaaaggagcctacagggaagcacaaggtttagtcagcttgctgaagagctgctgagaataatggctgcttttgagcttgacacgggaatgcagcttacaaatggttttagtttttcaaaaaagagaaataattcttgtgagcgtttgaatgaggaggcgtcgatcatccagagcgtgggctaccggaaccgtgtcagaaggcttccccaggtcgaacctggaaatgccaccttgaaggacagcctaggtgtccagctgtctaaccttggaatcgtgagatcagtgaagaaaaacaggcagacccaacctcgaaagaaatctgtctacattgaactagactctgattcttctgaagagacagtaactaagccaggtgattgcagtgtgagagaccaggaattgttacagaccgcccctcaagaagctggagatgaaggcaagctgcactctgcagaagaggctgcttgtgagttttctgagggcataagaaacattgaacatcatcaatgcagtgatgatttaaaccctactgagaatcatgcaactgaaaggcatccagaaaaatgtcagagtatttctatttcaaatgtgtgtgtggagccatgtggcacagatgctcatgccagctcattacagcctgagaccagcagtttattgctcattgaagacagaatgaatgcagaaaaggctgaattctgtaataaaagcaaacagcctggcatagcagtgagccagcagagcagatgggctgcaagtaaaggaacatgtaacgacaggcaggttcccagcactggggaaaaggtaggtccaaacgctgactcccttagtgatagagagaagtggactcacccgcaaagtctgtgccctgagaattctggagctaccaccgatgttccttggataacactaaatagcagcgttcagaaagttaatgagtggttttccagaactggtgaaatgttaacttctgacagcgcatctgccaggaggcacgagtcaaatgctgaagcagctgttgtgttggaagtttcaaacgaagtggatgggggttttagttcttcaaggaaaacagacttagtaacccccgacccccatcatactttaatgtgtaaaagtggaagagacttctccaaaccagtagaggataatatcagtgataaaatatttgggaaatcctatcagagaaagggaagccgccctcacctgaaccatgtgactgaaattataggcacatttattacagaaccacagataacacaagagcagcccttcacaaataaattaaaacgtaagagaagtacatcccttcaacctgaggacttcatcaagaaagcagattcagcaggtgttcaaaggactcctgacaacataaatcagggaactgacctaatggagccaaatgagcaagcagtgagtactaccagtaactgtcaggagaacaaaatagcaggtagtaatctccagaaagagaaaagcgctcatccaactgaatcattgagaaaggaacctgcttccacagcaggagccaaatctataagcaacagtgtaagtgatttggaggtagaattaaacgtccacagttcaaaagcacctaagaaaaataggctgaggaggaagtcttctatcaggtgtgctcttccacttgaaccaatcagtagaaatccaagcccacctacttgtgctgagcttcaaatcgatagttgtggtagcagtgaagaaacaaagaaaaaccattccaaccaacagccagccgggcaccttagagagcctcaactcatcgaagacactgaacctgcagcggatgccaagaagaacgagccaaatgaacacataaggaagagacgtgccagcgatgctttcccagaagagaaattaatgaacaaagctggtttattaactagctgttcaagtcctagaaaatctcaagggcctgtcaatcccagccctcagagaacaggaacagagcaacttgaaacacgccaaatgtctgacagtgccaaagaactcggggatcgggtcctaggaggagagcccagtggcaaaaccactgaccgatctgaggagagcaccagcgtatccttggtatctgacactgactacgacactcagaacagtgtctcagtcctggacgctcacactgtcagatatgcaagaacaggatccgctcagtgtatgactcagtttgtagcaagcgaaaaccccaaggaactcgtccatggctctaacaatgctgggagtggcacagagggtctcaagccccccttgagacacgcgcttaacctcagtcaggagaaagtagaaatggaagacagtgaacttgatactcagtatttgcagaatacatttcaagtttcaaagcgtcagtcatttgctttattttcaaaacctagaagtccccaaaaggactgtgctcactctgtgccctcaaaggaactgagtccaaaggtgacagctaaaggtaaacaaaaagaacgtcagggacaggaagaatttgaaatcagtcacgtacaagcagttgcggccacagtgggcttacctgtgccctgtcaagaaggtaagctagctgctgatacaatgtgtgatagaggttgtaggctttgtccatcatctcattacagaagcggggagaatggactcagcgccacaggtaaatcaggaatttcacaaaactcacattttaaacaatcagtttctcccatcaggtcatctataaaaactgacaataggaaacctctgacagagggacgatttgagagacatacatcatcaactgagatggcggtgggaaatgagaacattcttcagagtacagtgcacacagttagcctgaataacagaggaaatgcttgtcaagaagccggctcgggcagtattcatgaagtatgttccactggtgactccttcccaggacaactaggtagaaacagagggcctaaggtgaacactgtgcctccattagatagtatgcagcctggtgtctgtcagcaaagtgttcctgtaagtgataagtatcttgaaataaaaaagcaggagggtgaggctgtctgtgcagacttctctccacgtctattctcagaccatcttgagcaatctatgagtggtaaggtttttcaggtttgctctgagacacctgatgacctgctggatgatgttgaaatacagggacatactagctttggtgaaggtgacataatggagagatctgctgtctttaacggaagcatcctgagaagggagtccagtaggagccctagtcctgtaacccatgcatcgaagtctcagagtctccacagagcgtctaggaaattagaatcgtcagaagagagcgactccactgaggatgaagatcttccctgcttccaacacttactgagcagaataagcaacacacctgagcttaccagatgcagcagtgctgtgacacagcgtatgccagagaaagcggaggggacccaagcaccatggaagggtagcagcagtgactgcaataatgaggtgatcatgatagaggcatctcaggagcatcagtttagtgaggatccaagatgctctggcagcatgttctcttcacagcacagtgctgcccaagggtcaactgcaaatgcaaactcccaggattccaattttattccaccttccaaacagaggagtcaccagtgtgggaatgaggaagctttcctaagtgacaaggaattgatttcagataacgaggaaatggcaacttgcctagaagaggataatgaccaagaagaggatagtataatcccagattcagaggcatccggatacgagagtgaaacaaacctttctgaagactgctcgcagagtgatattttaaccactcagcagcgggcgaccatgaagtataacctgataaagctgcagcaggaaatggctcacctggaagctgtgctggagcagcgtgggaaccagccttctggccactccccttccctcctagcggacccttgtgccctggaagacctgccagatctggaaccaaacatgtcaggagcagcaattttaacttcaaagaacattaatgagaatcctgtaagccaaaatttgaagagcgcttgtgatgacaaattccaactacaacatctggagggtcccaccagtggagatgacgagtcagggatgggaaggccttccccttttaaatctccgttggcaggcagtaggggctctgcacatggctgctctaggcatcttcaaaagagaaactccccctctcaggaggagctcctccagcctgctggatcagaggcgtcatctgagccacacaattcaacagggcagtcttgcctgccaaggcgagagctagaaggaaccccatacctgggatctggaatcagccttttctctagtagagaccccgaatctgagtcccctaaagagccagcccacattggcaccacaccagcttcaacctctgcactgaaaataccccaaggtcaagttgctttccggagtgcagctgctgctggtgctgataaagcagtggtaggaattgtgagcaagataaagccggaattgacatcttcagaagaaagagcggatagagacatatccatggtggtgtcaggcttgacccccaaagaagtaatgaccgtgcaaaagtttgctgaaaaataccgcctcactttaactgacgcaattactgaggagactacacatgtaattataaaaacagatgcggagtttgtgtgtgagcggacactgaaatattttctgggcattgcaggaggaaagtggatagttagctattcatgggtggtccggtctatccaagaaagaagacttctgaatgtgcatgaatttgaagtcaaaggagatgttgtgactggaagaaatcaccaaggtccaaggcgatccagagaatcccgggaaaagctcttcaagggcctacaggtctattgttgtgagcccttcaccaacatgcccaaagatgagctggagaggatgctgcagctgtgtggggcttccgtggtgaaggagcttccatcgctcacccatgacacaggtgctcatctagttgtgatcgtgcagccaagcgcctggacagaagacagcaactgcccagatattgggcagctgtgcaaggcacgtcttgtgatgtgggactgggtgttggacagtctatccagctaccggtgtcggggtctggatgcctacctggtacagaatatcacctgtgacagtagtgagccacaagactccaatgattaatttaagaaatggtcttccatgcccggggagcacctctcagtctttggtgcttccacaccctacttactaagtatttttcatgcatccgcctggaaaaaccagcttctacctatgtgagggtgggtgccttaaaggttttctaaccaagtccccctttgaagtctgtcttgaacacaaaaagttttcacctgagaaagctt

SEQ ID NO: 14 - protein, 1812 aa, Mus musculus
MDLSAVQIQEVQNVLHAMQKILECPICLELIKEPVSTKCDHIFCKFCMLKLLNQKKGPSQCPLCKNEITKRSLQGSTRFSQLAEELLRIMAAFELDTGMQLTNGFSFSKKRNNSCERLNEEASIIQSVGYRNRVRRLPQVEPGNATLKDSLGVQLSNLGIVRSVKKNRQTQPRKKSVYIELDSDSSEETVTKPGDCSVRDQELLQTAPQEAGDEGKLHSAEEAACEFSEGIRNIEHHQCSDDLNPTENHATERHPEKCQSISISNVCVEPCGTDAHASSLQPETSSLLLIEDRMNAEKAEFCNKSKQPGIAVSQQSRWAASKGTCNDRQVPSTGEKVGPNADSLSDREKWTHPQSLCPENSGATTDVPWITLNSSVQKVNEWFSRTGEMLTSDSASARRHESNAEAAVVLEVSNEVDGGFSSSRKTDLVTPDPHHTLMCKSGRDFSKPVEDNISDKIFGKSYQRKGSRPHLNHVTEIIGTFITEPQITQEQPFTNKLKRKRSTSLQPEDFIKKADSAGVQRTPDNINQGTDLMEPNEQAVSTTSNCQENKIAGSNLQKEKSAHPTESLRKEPASTAGAKSISNSVSDLEVELNVHSSKAPKKNRLRRKSSIRCALPLEPISRNPSPPTCAELQIDSCGSSEETKKNHSNQQPAGHLREPQLIEDTEPAADAKKNEPNEHIRKRRASDAFPEEKLMNKAGLLTSCSSPRKSQGPVNPSPQRTGTEQLETRQMSDSAKELGDRVLGGEPSGKTTDRSEESTSVSLVSDTDYDTQNSVSVLDAHTVRYARTGSAQCMTQFVASENPKELVHGSNNAGSGTEGLKPPLRHALNLSQEKVEMEDSELDTQYLQNTFQVSKRQSFALFSKPRSPQKDCAHSVPSKELSPKVTAKGKQKERQGQEEFEISHVQAVAATVGLPVPCQEGKLAADTMCDRGCRLCPSSHYRSGENGLSATGKSGISQNSHFKQSVSPIRSSIKTDNRKPLTEGRFERHTSSTEMAVGNENILQSTVHTVSLNNRGNACQEAGSGSIHEVCSTGDSFPGQLGRNRGPKVNTVPPLDSMQPGVCQQSVPVSDKYLEIKKQEGEAVCADFSPRLFSDHLEQSMSGKVFQVCSETPDDLLDDVEIQGHTSFGEGDIMERSAVFNGSILRRESSRSPSPVTHASKSQSLHRASRKLESSEESDSTEDEDLPCFQHLLSRISNTPELTRCSSAVTQRMPEKAEGTQAPWKGSSSDCNNEVIMIEASQEHQFSEDPRCSGSMFSSQHSAAQGSTANANSQDSNFIPPSKQRSHQCGNEEAFLSDKELISDNEEMATCLEEDNDQEEDSIIPDSEASGYESETNLSEDCSQSDILTTQQRATMKYNLIKLQQEMAHLEAVLEQRGNQPSGHSPSLLADPCALEDLPDLEPNMSGAAILTSKNINENPVSQNLKSACDDKFQLQHLEGPTSGDDESGMGRPSPFKSPLAGSRGSAHGCSRHLQKRNSPSQEELLQPAGSEASSEPHNSTGQSCLPRRELEGTPYLGSGISLFSSRDPESESPKEPAHIGTTPASTSALKIPQGQVAFRSAAAAGADKAVVGIVSKIKPELTSSEERADRDISMVVSGLTPKEVMTVQKFAEKYRLTLTDAITEETTHVIIKTDAEFVCERTLKYFLGIAGGKWIVSYSWVVRSIQERRLLNVHEFEVKGDVVTGRNHQGPRRSRESREKLFKGLQVYCCEPFTNMPKDELERMLQLCGASVVKELPSLTHDTGAHLVVIVQPSAWTEDSNCPDIGQLCKARLVMWDWVLDSLSSYRCRGLDAYLVQNITCDSSEPQDSND

SEQ ID NO: 15 - DNA, 5711 bp, Homo sapiens
agctcgctgagacttcctggaccccgcaccaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 16 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 17 - DNA, 7041 bp, Homo sapiens
atggatgaaccatctcccttggcccaacctctggagctgaaccagcactctcgattcataataggttctgtgtcagaagataactcagaggatgagatcagcaacctggtgaagctggacctactggaggagaaggagggctccttgtcacctgcttctgttggctcagatacactctctgatttggggatctctgccctacaggatggcttggccttgcacataaggtccagctggtctggcttgcacctagtaaagcagggcggagacagaaagaaaatagattctcaacgagatttcactgtggcttctccagcagaatttgttactcgctttgggggaaataaagtgattgagaaggttctcatcgctaacaatggcattgcagcagtgaaatgcatgcggtctatccgtaggtggtcttatgaaatgtttcgaaatgaacgtgcaattagattcgttgtcatggtcacacctgaagaccttaaagccaatgcagaatacattaagatggcagatcactatgtgccggtgcctggaggagcaaacaacaacaactatgcaaatgtggaattaattcttgatattgctaaaaggatcccagtgcaagcagtgtgggctggctggggtcatgcttctgagaatcccaaactaccggaacttctcttgaaaaatggcattgccttcatgggtcctccaaaccaggccatgtgggctttaggggataagattgcatcttccatagtggctcaaactgcaggtatcccaactcttccctggagcggcagtggtcttcgtgtggactggcaggaaaatgatttttcaaaacgtatcttaaatgttccccaggagctatatgaaaaaggttatgtgaaagatgtggatgatgggctaaaggcagctgagaaggttggatatccagtaatgatcaaggcctcagagggaggaggagggaagggaattagaaaagttaacaatgcagatgacttccctaatctcttcagacaggttcaagctgaagttcctggatctcccatatttgtgatgagactagccaaacaatctcgtcatctggaggtgcagatcttagcggaccaatatggcaatgctatctctttgtttggtcgtgattgctctgtacaacgcaggcatcagaagattattgaagaagcacctgctactattgctactccagcagtatttgaacacatggaacagtgtgcggtgaaacttgccaaaatggtgggttatgtgagtgctgggactgtggaatacctgtacagccaggatggcagcttctactttctagaattgaaccctcggctacaggttgaacaaccttgtacagagatggtggctgatgtcaatctccccgcagcacagctccagattgccatggggattcctctatatagaatcaaggatatccgtatgatgtatggggtatctccttggggtgattctcccattgattttgaaaattctgctcacgttccttgtccaaggggccatgttattgctgctcggatcactagtgaaaatccagatgagggttttaagcccagctcaggaacagttcaggagctaaatttccgcagcaataagaatgtttggggatatttcagtgttgctgctgcagggggacttcatgaatttgctggttctcagtttggtcactgcttttcttggggagaaaacagagaagaagcaatttcaaacatggtggtggcattgaaggagctgtctattcggggtgactttcgaactacagttgaatacctgatcaaattgttagagactgaaagctttcaaatgaacagaattgatactggctggctggacagactgatagcagaaaaagtacgggctgagcgtcctgacaccatgttgggggttgtgtgtggtgccctccacgtcggagatgtgagcctgcgaaatagcgtctctaacttccttcactccttagaaaggggtcaagtccttccggctcatacacttctgaatacagtagatgttgaacttatctatgagggagtcaagtatgtacttaaggtgactcgacagtcccccaactcctatgtggtgatcatgaatggctcatgtgtagaagtagatgtacatcggctgagtgacggtggactgctcttgtcctatgatggcagcagttacaccacgtatatgaaggaggaagtagacagatatcgcatcacaattggcaataaaacctgtgtgtttgagaaggaaaatgacccatcggtgatgcgctcaccttctgctgggaagttaatccagtacattgtagaagatggaggtcatgtgtttgccggccagtgctatgcagagattgaggtaatgaagatggtaatgactttgacagctgtggagtctggctgtatccattacgtcaagcgtcctggagcagctcttgaccctggctgtgtactcgccaaaatgcaactggacaaccccagcaaggttcagcaggctgaacttcacacaggtagtctgccacggatccagagcaccgctctccgaggcgagaagctccatcgagtgttccactatgtcctggataatctggtcaatgtaatgaatggatactgccttccagatcctttctttagcagcaaggtaaaagactgggtagaacgattgatgaaaaccctcagagatccctccctgcctctcctagaattgcaagatattatgaccagtgtgtctggccgcattccccccaatgtggagaagtctatcaagaaggaaatggctcagtatgctagcaacatcacatcagtcctctgtcagtttcccagccagcagattgcaaacatcctagatagccatgcagctacattgaaccggaaatctgaacgggaagtcttctttatgaatactcagagcattgtccagctggtacagaggtaccgaagtggcatccgaggccacatgaaggctgtggtgatggatctgctccggcagtacctgcgagtagagacacaattccagaatggtcactatgacaaatgtgtattcgcccttcgagaagagaataagagtgacatgaacactgtactgaactacatcttctctcacgctcaagtcaccaagaagaatcttctggtcacaatgcttattgatcagttgtgtggccgggaccctactctaactgatgagctgctgagtattctcacagagctaactcaactcagtaagaccaccaatgccaaagtagcacttcgagcacgccaggttcttattgcctcccatttgccatcatatgacgttcgccataaccaagtagagtctatcttcctatcagctattgacatgtatggacatcaattttgcattgagaacctgcagaaactcatcctatcagaaacatctatttttgatgtcctaccaaacttcttctatcacagcaaccaagtagtgaggatggcagctctggaggtgtacgttcgaagggcttatattgcctatgaacttaacagcgtacaacaccgccagcttaaggacaacacctgcgtggtggaattccagttcatgctgcccacatctcatccaaacagagggaacatccctacgctaaacagaatgtccttctcctccaacctcaaccactatggcatgacccatgtagctagtgtcagcgatgttctgttggacaacgccttcacaccaccttgtcaacgcatgggcggaatggtctcttttcggacttttgaagattttgtcaggatctttgatgaagtaatgggctgcttctgtgactccccaccccagagtcccacattccctgaggcaggtcacacgtctctttatgatgaggataaggttcccagggatgaaccaattcacattctcaatgtggctatcaagactgacggtgatattgaggatgacaggctggcagctatgttcagagaattcacccagcaaaataaagctaccctggctgaccatgggatccggcgcctgactttcctggttgcacaaaaggatttcagaaagcaggtcaactatgaggtggatcggagatttcatagagaattccctaaattttttacattccgagcaagggataagtttgaggaggatcgtatctatcgtcatctggagcctgctctggctttccagttagagctgaaccggatgagaaattttgacctcactgccattccatgtgctaatcacaagatgcacctgtatctcggggcagccaaggtggaagtgggcacagaagtgacagactacaggttctttgttcgtgcaatcatcaggcattctgatctggtcaccaaggaagcttcttttgaatatctgcaaagtgaaggggagcggctactcctggaagccatggatgagttggaagttgcttttaacaatacaaatgtccgcactgactgtaaccacatcctcctcaactttgtgcccacggttatcatggacccatcaaagattgaggaatccgtgcggagcatggtaatgcggtatggaagtcgcctgtggaaattgcgcgtcctccaggcagaactgaaaatcaacattcgcctgacgccaactggaaaagcaattcccatccgccttttcctgacaaacgagtctggctattacttggatatcagcctatacaaggaagtgactgactccaggacagcacagatcatgtttcaggcatatggagacaagcagggaccactgcatggaatgttaatcaatactccatatgtgaccaaagacctgctgcaatcaaagaggttccaggcacaatccttagggacaacgtacatatatgatatcccagagatgtttcggcagtccctgatcaaactctgggagtctatgtcaactcaagcatttcttccatctccccctctgccttctgacatgctgacttacactgaactggtactggatgatcaaggtcagctggtccacatgaacaggcttccaggaggaaatgagattggcatggtagcttggaaaatgagccttaaaagtcctgaatatccagaaggccgagatgttattgttattggcaatgacattacataccgaattgggtcctttgggcctcaagaagatttgttatttctcagagcttccgaacttgctagggccgaaggcattccacgcatctatgtatcagccaacagtggagcaagaatcggactggcagaagaaattcgccatatgtttcatgtggcctgggtagattctgaggatccttacaagggatacaggtatttatatctgactcctcaagattataagagagtcggtgctctcaactctgtccattgtgaacacgtggaagatgaaggagaatccaggtacaagataacagatattattgggaaagaagagggaattggacccgagaaccttcgaggttctggaatgatcgctggagaatcctcattggcctataatgagatcattaccatcagcctggtgacgtcccgggccattgggattggggcttaccttgtccggctgggacagagaaccatccaggttgagaattctcacttgattctaacaggagctggagccctcaacaaagtcctcgggcgggaagtgtacacctccaataaccagctggggggcatccagattacgcacaacaatggggtgacccactgcactgtgtgtgacggctttgaaggggttttcactgtcctgcactggctgtcttacatgcccaagagcgtacacagttcagttcctcttctgaactcaaaggatcctatagacagaatcatcgagtttgttcccacaaagaccccatatgatcctcgatggatgctagcaggccgccctcacccaacccaaaaaggtcagtggttgagtggcttttttgactatggatctttctcagagattatgcagccctgggcacagacggtggtggttggtagagccaggttagggggaatacctgtgggagttgttgctgtagaaacccggacagtagaactaagtgtaccagctgatccagcaaacctggattctgaagccaagataatccagcacgccggccaagtttggtttccggattctgcgtttaagacgtatcaggccatcaaggacttcaaccgggaagggctacctctaatggtctttgccaactggagaggcttctctggtggaatgaaagatatgtatcaccaagtgctgaagtttggtgcttacattgtggatggcttgcgggaatgttcccagcctgtgctggtctacattcctccccaggctgagctgcggggtggctcctgggtggtgatcgacccaaccatcaatcctcggcacatggagatgtatgctgaccgagaaagcaggggatctgttctggagccagaagggacggtagaaatcaaattccgcagaaaggatctggtgaaaaccatgcgtcgggtggacccagtctacatccacttggctgagcgattggggaccccagagctgagcccaactgagcggaaggagttggagagcaagttgaaggagcgggaggaattcctaattccaatttaccatcaggtagccgtgcagtttgctgacttgcacgacactccaggccggatgcaggagaagggtgttattagcgatatcctggattggaaaacatcccgtaccttcttctactggcgactgaggcgtcttctgctggaggacctggtcaagaagaaaatccacagtgccaaccctgagctgactgatggccagattcaagccatgttaagacgctggtttgtggaagtggaaggaacagtgaaggcttatgtttgggacaataataaggatctggcggagtggctagagaaacagctgacagaggaggatggtgttcactcggtaatagaggaaaacatcaaatgcatcagcagagactacgtcctcaagcaaatccgcagcttggtccaggccaatccagaggttgccatggattccatcatccatatgacgcagcacatatcacccactcagcgggcagaagtcataaggatcctttccactatggactccccttctacgtag

SEQ ID NO: 18 - protein, 2346 aa, Homo sapiens
MDEPSPLAQPLELNQHSRFIIGSVSEDNSEDEISNLVKLDLLEEKEGSLSPASVGSDTLSDLGISALQDGLALHIRSSWSGLHLVKQGGDRKKIDSQRDFTVASPAEFVTRFGGNKVIEKVLIANNGIAAVKCMRSIRRWSYEMFRNERAIRFVVMVTPEDLKANAEYIKMADHYVPVPGGANNNNYANVELILDIAKRIPVQAVWAGWGHASENPKLPELLLKNGIAFMGPPNQAMWALGDKIASSIVAQTAGIPTLPWSGSGLRVDWQENDFSKRILNVPQELYEKGYVKDVDDGLKAAEKVGYPVMIKASEGGGGKGIRKVNNADDFPNLFRQVQAEVPGSPIFVMRLAKQSRHLEVQILADQYGNAISLFGRDCSVQRRHQKIIEEAPATIATPAVFEHMEQCAVKLAKMVGYVSAGTVEYLYSQDGSFYFLELNPRLQVEQPCTEMVADVNLPAAQLQIAMGIPLYRIKDIRMMYGVSPWGDSPIDFENSAHVPCPRGHVIAARITSENPDEGFKPSSGTVQELNFRSNKNVWGYFSVAAAGGLHEFAGSQFGHCFSWGENREEAISNMVVALKELSIRGDFRTTVEYLIKLLETESFQMNRIDTGWLDRLIAEKVRAERPDTMLGVVCGALHVGDVSLRNSVSNFLHSLERGQVLPAHTLLNTVDVELIYEGVKYVLKVTRQSPNSYVVIMNGSCVEVDVHRLSDGGLLLSYDGSSYTTYMKEEVDRYRITIGNKTCVFEKENDPSVMRSPSAGKLIQYIVEDGGHVFAGQCYAEIEVMKMVMTLTAVESGCIHYVKRPGAALDPGCVLAKMQLDNPSKVQQAELHTGSLPRIQSTALRGEKLHRVFHYVLDNLVNVMNGYCLPDPFFSSKVKDWVERLMKTLRDPSLPLLELQDIMTSVSGRIPPNVEKSIKKEMAQYASNITSVLCQFPSQQIANILDSHAATLNRKSEREVFFMNTQSIVQLVQRYRSGIRGHMKAVVMDLLRQYLRVETQFQNGHYDKCVFALREENKSDMNTVLNYIFSHAQVTKKNLLVTMLIDQLCGRDPTLTDELLSILTELTQLSKTTNAKVALRARQVLIASHLPSYDVRHNQVESIFLSAIDMYGHQFCIENLQKLILSETSIFDVLPNFFYHSNQVVRMAALEVYVRRAYIAYELNSVQHRQLKDNTCVVEFQFMLPTSHPNRGNIPTLNRMSFSSNLNHYGMTHVASVSDVLLDNAFTPPCQRMGGMVSFRTFEDFVRIFDEVMGCFCDSPPQSPTFPEAGHTSLYDEDKVPRDEPIHILNVAIKTDGDIEDDRLAAMFREFTQQNKATLADHGIRRLTFLVAQKDFRKQVNYEVDRRFHREFPKFFTFRARDKFEEDRIYRHLEPALAFQLELNRMRNFDLTAIPCANHKMHLYLGAAKVEVGTEVTDYRFFVRAIIRHSDLVTKEASFEYLQSEGERLLLEAMDELEVAFNNTNVRTDCNHILLNFVPTVIMDPSKIEESVRSMVMRYGSRLWKLRVLQAELKINIRLTPTGKAIPIRLFLTNESGYYLDISLYKEVTDSRTAQIMFQAYGDKQGPLHGMLINTPYVTKDLLQSKRFQAQSLGTTYIYDIPEMFRQSLIKLWESMSTQAFLPSPPLPSDMLTYTELVLDDQGQLVHMNRLPGGNEIGMVAWKMSLKSPEYPEGRDVIVIGNDITYRIGSFGPQEDLLFLRASELARAEGIPRIYVSANSGARIGLAEEIRHMFHVAWVDSEDPYKGYRYLYLTPQDYKRVGALNSVHCEHVEDEGESRYKITDIIGKEEGIGPENLRGSGMIAGESSLAYNEIITISLVTSRAIGIGAYLVRLGQRTIQVENSHLILTGAGALNKVLGREVYTSNNQLGGIQITHNNGVTHCTVCDGFEGVFTVLHWLSYMPKSVHSSVPLLNSKDPIDRIIEFVPTKTPYDPRWMLAGRPHPTQKGQWLSGFFDYGSFSEIMQPWAQTVVVGRARLGGIPVGVVAVETRTVELSVPADPANLDSEAKIIQHAGQVWFPDSAFKTYQAIKDFNREGLPLMVFANWRGFSGGMKDMYHQVLKFGAYIVDGLRECSQPVLVYIPPQAELRGGSWVVIDPTINPRHMEMYADRESRGSVLEPEGTVEIKFRRKDLVKTMRRVDPVYIHLAERLGTPELSPTERKELESKLKEREEFLIPIYHQVAVQFADLHDTPGRMQEKGVISDILDWKTSRTFFYWRLRRLLLEDLVKKKIHSANPELTDGQIQAMLRRWFVEVEGTVKAYVWDNNKDLAEWLEKQLTEEDGVHSVIEENIKCISRDYVLKQIRSLVQANPEVAMDSIIHMTQHISPTQRAEVIRILSTMDSPST

SEQ ID NO: 19 - protein, 11 aa, Mus musculus
VEVGTEVTDYR

SEQ ID NO: 20 - protein, 13 aa, Mus musculus
EASFEYLQNEGER

SEQ ID NO: 21 - protein, 7 aa, Mus musculus
DFTVASP

SEQ ID NO: 22 - protein, 9 aa, Mus musculus
KAAEEVGYP

SEQ ID NO: 23 - protein, 9 aa, Mus musculus
KDLVEWLEK

(19) United States
(12) Patent Application Publication
(10) Pub. No.: US 2006/0105403 A1
(43) Pub. Date: May 18, 2006

(54) Braca1/acc alpha molecular complexes, diagnostic and therapeutic applications

(76) Inventors: Nicole Dalla Venezia, Lyon (FR); Clemence Magnard, Lyon (FR); Gilbert Lenoir, Lyon (FR); Olga Sinilnikova-Erard, Millery (FR)

Correspondence Address: YOUNG & THOMPSON, 745 SOUTH 23RD STREET, 2ND FLOOR, ARLINGTON, VA, 22202, US

(21) Appl. No.: 10/480,434

(22) PCT Filed: Jun. 12, 2002
(86) PCT No.: PCT/FR02/02016

§ 371 Date: Aug. 2, 2004

(30) Foreign Application Priority Data
Jun. 13, 2001 (FR) 01/07740
Mar. 5, 2002 (FR) 02/02789

(51) Int. Cl. G01N 33/574; C07K 14/82; C07K 16/30

(52) U.S. Cl. 435/7.23; 530/350; 530/388.8

(57) ABSTRACT

The invention concerns a molecular complex comprising: a first polypeptide including the sequence of amino acids 1640 to 1863 the human BRCA1 protein or a similar sequence of amino acids of the protein BRCA1 in another animal species, and a second polypeptide including a fragment of the ACC-α protein capable of binding with said first protein.

The invention relates to a protein-protein complex implicated in the predisposition to cancers of the breast and of the ovary. More particularly this protein-protein interaction has applications for the screening of molecules having a therapeutic activity in the treatment and the prevention of cancer, as well as diagnostic applications.

Cancer of the breast is a very common disease which affects almost 10% of women in the western world. There are two forms of this cancer: a majority sporadic form and a familial form concerning 5 to 10% of cases. In predisposed families, the risk is transmitted in a dominant autosomal manner (Ford D. et al, 1998), and the mammary tumours are frequently associated with other cancers, particularly cancer of the ovary. The study of predisposed families has made it possible to identify two major genes for predisposition to breast cancer: BRCA1 (Breast Cancer 1), localised at 17q21 (Miki Y. et al, 1994) and BRCA2, localised at 13q12-13 (Wooster R. et al, 1995). The majority of familial cases of cancer of the breast and/or of the ovary are linked to mutations of the gene BRCA1 (Ford D. et al, 1998). The gene BRCA1 extends over almost 81 kb (Smith T. M. et al, 1996) and codes for a ubiquitous transcript of 7.8 kb translated into a protein of 1863 amino acids. The gene BRCA1 is very conserved, its murine homologue codes for a protein of 1812 amino acids which is 58% identical to the human BRCA1 protein (Bennett L. M. et al, 1995).

The function of the protein BRCA1 is still little known and the study of its cellular localisation has been the subject of controversy. According to the most recent studies, BRCA1 is a nuclear protein of 220 kDa (Scully R. et al, 1997a). The form shortened by splicing of the exon 11, an exon which contains two sequences coding for nuclear localisation signals, is itself a cytoplasmic protein of 110 kDa Despite the large size of this protein, only two remarkable domains have been evidenced: a domain in zinc fingers at the amino-terminal end (Miki Y. et al, 1994) and a BRCT domain (BRCA1 C-terminus) at the carboxy-terminal end (Koonin E. V. et al, 1996; Callebaut I. and Mornon J. P., 1997; Bork P. et al, 1997).

The current data on the function of the BRCA1 protein suggest a role in different biological mechanisms:

1) a frequent loss of heterozygosity concerning the BRCA1 wild allele has been demonstrated in mammary tumours (Smith A. A. et al, 1992), suggesting that BRCA1 can act as a tumour suppressor;

2) the BRCA1 protein seems to play an essential part in the course of embryonic development and the differentiation of the mammary gland in the mouse (Hakem R. et al, 1995) and also in the human (Magdinier F. et al, 1999);

3) BRCA1 would be a transactivator of transcription (Chapman M. S. and Verma I. M., 1996; Monteiro A. N. A. et al, 1996) and would be associated with RNA polymerase II (Scully R. et al, 1997b);

4) BRCA1 would be implicated in the DNA repair pathways (Scully R et al, 1997c; Haken R. et al, 1997; Ouchi T. et al, 1998) and the control of the cell cycle (Larson J. S. et al, 1997);

5) Finally, several known proteins have been identified as being partners of BRCA1, including BARD1 (Wu L. C. et al, 1996), p53 (Zhang H. et al, 1998a), c-Myc (Wang, Q. et al, 1998), Rad 51 (Scully R. et al, 1997a), BRCA2 (Chen J. et al, 1998), CtIP (Yu X. et al, 1998), RNA helicase A within the RNA polymerase II holoenzyme complex (Anderson S. F. et al, 1998), the histone deacetylase complex (Yarden R. et al 1999), or the protein BACH1 (Cantor S. B. et al, 2001); their regions of interaction are distributed over all of the BRCA1 protein.

In 1996, a search for sequence similarity between the BRCA1 protein and the known proteins of databases made it possible to demonstrate a region of interest situated at the C-terminal end of the BRCA1 protein: the domain BRCT (BRCA1 C-terminal) (Koonin E. V. et al, 1996). A more detailed study of this region by the “Hydrophobic Cluster Analysis” (HCA) technique (Callebaut I. and Mornon J. P., 1997) permitted characterisation of the BRCT domain. It consists of the duplication of a module of about a hundred amino acids having very conserved sequences. This BRCT module is found within about forty proteins present in various species (Bork P. et al, 1997). Amongst the proteins with BRCT of which the function is characterised, the majority are implicated in the mechanisms for DNA repair and for cell cycle control.

In certain of these proteins, the BRCT module seems to play a major part in the mediation of protein interactions (XRCCA/DNA ligase IV (Critchlow S. E. et al, 1997), XRCC1/DNA ligase III (Nash R. et al, 1997) and XRCC1/PARP (Masson M. et al, 1998) complexes). It would also appear to be responsible for the homodimerisation of the XRCC1 protein (Zhang X. et al, 1998b). In the BRCA1 protein, the BRCT domain is particularly well conserved: the identity of sequence with the murine form is 72% for this region as against only 58% for the total protein (Bennett L. M. et al, 1995), and its globular structure is conserved. The majority of the deleterious germinal mutations do not permit the synthesis of this region of BRCA1. Furthermore, a large number of false-sense mutations directly affect this domain. This C-terminal part also seems essential to the normal function of the protein: its suppression leads to withdrawing from BRCA1 its capacity to inhibit the growth of cancerous cells as well as its function as transcription transactivator. Finally, it has already been possible to identify protein partners of BRCA1 which interact specifically with its C-terminal end containing the BRCT domain: BRCA2, CtIP, RNA helicase A and the histone deacetylase complex, the BACH1 protein.

In order to identify proteins which interact with the tandem formed from the two BRCT modules of Brca1, the inventors carried out a co-sedimentation test in a lysate of murine fibroblasts, with a GST fusion protein containing amino acids 1583 to 1812 of murine Brca1, called GST-BRCT. The analysis of the protein complexes co-sedimentation showed that a major band of 210 kDa was visible only with the specimen GST-BRCT. The inventors have shown that the tandem of the two BRCT domains was necessary and sufficient for the formation of this complex. Moreover, the inventors have shown that the formation of this complex was conserved in the human cells, using a GST-human BRCT fusion protein in co-sedimentation experiments similar to those cited above.

Thus the work by the inventors relating to the functional role of BRCA1 have enabled them to bring to light a new protein-protein interaction which implicates, on the one hand, the BRCA1 protein and more particularly the BRCT domain situated at the carboxy-terminal end thereof, and, on the other hand, a known protein, acetyl-Coenzyme-A carboxylase, α form (ACC-α).

The BRCA1 protein and the gene coding for this protein are described in particular in Patent Applications EP 699 754 and EP 705 902. The sequence SEQ ID No. 13 corresponds to the cDNA of the murine Brca1 gene (Genbank: MMU31625), SEQ ID No. 14 corresponds to the amino acid sequence of murine Brca1, SEQ ID No. 15 corresponds to the cDNA of the human BRCA1 gene (Genbank HSU14680), and SEQ ID No. 16 corresponds to the amino acid sequence of human BRCA1.

ACC-α is a cytoplasmic protein of 265 kDa of which the enzymatic activity plays a key role in the synthesis pathway of long-chain fatty acids.

The cDNA of human ACC-α (SEQ ID No. 17) was cloned in 1995 by Abu-Elheiga et al. Its sequence is very conserved in human, rat, chicken and in yeast, in particular at the sites for binding to biotin, to ATP and to coenzyme A. The protein sequence of human ACC-α is indicated by SEQ ID No. 18.

Co-immunoprecipitation experiments with an anti-Brca1 antibody on Bosc cellular lysates transfected by the wild form of the murine Brca1 protein or its spliced form Δ11 have been carried out. They have made it possible to confirm that the two forms of the murine protein combine with ACC-α in vivo. In the same way the entire human BRCA1 protein coprecipitates with the endogenous ACC-α of transfected Bosc cells.

A polyclonal antibody directed against the amino-terminal (MDEPSPLAQPLELNQ) (SEQ ID No. 1) and carboxy-terminal (AEVIRILSTMDSPST) (SEQ ID No. 2) ends of the human ACC-α protein was prepared in the rabbit and immunopurified against these two peptide sequences. Co-immunoprecipitation experiments with this anti-ACC-α antibody, called L3J74, on lysates of human mammary epithelial cells HBL100 showed in vivo, under physiological conditions, the existence of the endogenous BRCA1/ACC-α complex.

In order to specify the interaction domain of ACC-α on BRCA1, different GST-BRCT fusion proteins were prepared and tested by co-sedimentation on lysates of murine fibroblasts NIH3T3, and made it possible to show that a longer form of the murine BRCT domain (residues 1512-1812 of SEQ ID No. 14), like the complete BRCT domain tested in the first place (residues 1583-1812 of SEQ ID No. 14), interact with ACC-α. On the other hand, proteins for fusion to GST containing either the BRCT A module alone or the BRCT B module alone are incapable of combining with ACC-α. A GST fusion protein expressing the 220 amino-terminal residues of the murine Brca1 protein has enabled confirmation of the specificity of the interaction of ACC-α with the carboxy-terminal end of BRCA1.

Numerous familial mutations predisposing to breast cancer have been localised at the carboxy-terminal region of BRCA1. In order to measure the effect of such mutations on the ACC-α/BRCA1 interaction, fusion proteins were prepared and tested in co-sedimentation. The first mutations chosen in the human BRCA1 protein, R1835X and Y1853X, are situated in the most distal BRCT domain. They introduce a stop codon preventing the synthesis of the last 29 and 11 amino acids respectively of the human protein which therefore has an incomplete BRCT domain. The GST fusion protein containing the mutation W1777X, which mimics the human germinal mutation R1835X on the murine protein, also abolishes the interaction of murine Brca1 with ACC-α. In a similar manner, the mutations A1708E, P1749R and M1775R, which create an amino acid substitution in the BRCT A domain, in the sequence for binding the two BRCT domains and in the BRCT B domain respectively, destroy the bond of human BRCA1 to ACC-α.

Taking into account the important role of the C-terminal end of the BRCA1 protein in the predisposition to breast cancer, the BRCA1/ACC-α interaction can be exploited advantageously for the development of therapeutically useful molecules.

The invention therefore relates to a molecular complex comprising:

a first polypeptide comprising the sequence of amino acids 1640 to 1863 of the human BRCA1 protein or the same sequence of amino acids of the BRCA1 protein in another species of animal, and

a second polypeptide comprising a fragment of the ACC-α protein capable of binding to said first polypeptide.

The region defined by the residues of amino acids 1640 to 1863 of the human BRCA1 protein is called the “minimal BRCA1 domain”.

The term “same sequence of amino acids of the BRCA1 protein in another species of animal” is used to designate an orthologue of the domain defined by the amino acids 1640 to 1863 of the human BRCA1 protein.

Conventionally, two orthologous genes or two orthologous proteins are homologues appearing by specification, i.e. orthologous refers to the same gene or the same protein in two different species.

Preferably, the said same sequence of amino acids of the BRCA1 protein in another species of animal is a sequence from a non-human mammal, and in particular the sequence of amino acids 1583 to 1812 of the murine BRCA1 protein. Thus the domain defined by the amino acids 1583 to 1812 of the murine BRCA1 protein constitutes an orthologue of the minimal domain of the human BRCA1 protein.

“ACC-α protein” is understood to mean the human ACC-α protein or the corresponding protein of an animal species, particularly of a non-human mammal, especially the mouse.

A fragment of the ACC-α protein capable of binding the minimal human BRCA1 domain or the corresponding domain of an animal species, or a polypeptide comprising the said domain, constitutes a “minimal domain of the ACC-α protein”. The identification of the minimal domain of the ACC-α protein falls within the routine work of the person skilled in the art. This domain can be identified by conventional techniques such as the production of truncated forms of the ACC-α protein and co-immunoprecipitation experiments, for example in the presence of the domain of amino acids 1640 to 1863 of the human BRCA1 protein. A minimal domain of the ACC-α protein includes in particular the entire ACC-α protein.

The invention also relates to a method of screening of molecules capable of modulating the interaction between BRCA1 and ACC-α, that is to say capable of preventing or favouring the formation of the complex or wholly or partially breaking up the complex formed.

Therefore the invention relates to a method of screening, particularly in vitro, of molecules useful for the prevention or treatment of cancer of the breast and/or of the ovary in which the molecules are tested for their capacity to modulate the interaction between the BRCA1 and ACC-α proteins.

A method of screening according to the present invention comprises:

a) contacting, in any order whatsoever, two different partners or three different partners selected from the group consisting of a first polypeptide comprising the sequence of amino acids 1640 to 1863 of the human BRCA1 protein or the same sequence of amino acids of the BRCA1 protein, a second polypeptide comprising a fragment of the ACC-α protein capable of binding the said first polypeptide, and a candidate molecule;

b) the incubation of the said partners for a sufficient time to permit any interaction thereof;

c) in the case where only two different partners have been selected at step a), the addition of the third partner selected from the said group and incubation for a sufficient time to permit any interaction thereof;

d) determination of the capacity of the candidate molecule to modulate the interaction between BRCA1 and ACC-α.

Such a method more particularly permits identification of the molecules capable of modulating the interaction between BRCA1 and ACC-α.

The determination of the capacity of the candidate molecule to modulate the interaction between BRCA1 and ACC-α can be carried out with the aid of appropriate separation and/or detection means which are well known to the person skilled in the art. For example, the number of complexes formed in the presence of increasing concentrations of the candidate molecule could be quantified. This can be carried out in particular with the aid of conventional analysis techniques such as chromatography, electrophoresis, immunological labelling possibly in association with detectable labels such as fluorescent, isotopic or chromogenic labels. Reference may be made for example to Current Protocols in Molecular Biology, edited by F. M. Ausubel et al (John Wiley and Sons).

According to this method, a polypeptide comprising the minimal domain of BRCA1, a polypeptide comprising the minimal domain of ACC-α and at least one candidate molecule can be contacted and incubated for a sufficient time to permit their interaction, and possibly the formation or the breakdown of the BRCA1/ACC-α complex.

According to another aspect, at least one candidate molecule and a polypeptide comprising the minimal domain of BRCA1 or of ACC-α are contacted and incubated for a sufficient time to permit any interaction thereof. The minimal domain of BRCA1 or of ACC-α which is missing is then added and the whole is incubated for a sufficient time to permit the interaction of all of the elements, and possibly the formation of the BRCA1/ACC-α complex.

According to yet another variant, a polypeptide comprising the minimal domain of BRCA1 and a polypeptide comprising the minimal domain of ACC-α are pre-incubated in such a way as to permit the formation of the BRCA1/ACC-α complex before the addition of at least one candidate molecule and the incubation of the whole for a sufficient time to permit their interaction and possibly the breakdown of the BRCA1/ACC-α complex.

A preferred embodiment of this method comprises the following steps:

a) contacting a first polypeptide comprising the sequence of amino acids 1640 to 1863 of the human BRCA1 protein or the same sequence of amino acids of the BRCA1 protein with a second polypeptide comprising a fragment of the ACC-α protein capable of binding the said first polypeptide, one at least of the polypeptides being labelled in a detectable manner;

b) addition of a candidate molecule capable of modulating the interaction;

c) incubation of the said polypeptides in the presence of the candidate molecule under conditions and for a period of time which are sufficient for binding between the said polypeptides to take place;

d) quantification of the number of labelled molecules bound in the presence of increasing concentrations of the candidate molecule.

Amongst these molecules, some are agonists and others are antagonists of the BRCA1/ACC-α interaction.

Therefore the present invention proposes a method of screening of molecules capable of modulating the interaction between BRCA1 and ACC-α, characterised in that the said molecule is an antagonist of the BRCA1/ACC-α interaction.

According to another aspect, the present invention proposes a method of screening of molecules capable of modulating the interaction between BRCA1 and ACC-α, characterised in that the said molecule is an agonist of the BRCA1/ACC-α interaction.

“Antagonist” is understood to mean a molecule, the action of which opposes the formation or the maintenance of the BRCA1/ACC-α complex. In other words, the antagonist molecules according to the invention include for example inhibitors capable of preventing or limiting the interaction between BRCA1 and ACC-α or molecules capable of breaking down the complex when it is formed.

An “agonist” is understood to mean a molecule, the action of which contributes to or increases the formation of the BRCA1/ACC-α complex.

According to another aspect, the present invention relates to a method of identification ex vivo of molecules constituting endogenous ligands of the BRCA1/ACC-α complex. The term ligand has the common meaning of a molecule capable of interacting with the BRCA1/ACC-α complex. More particularly, a ligand may constitute an effector of the biological activity of the BRCA1/ACC-α complex. In this sense, a cellular receptor of the BRCA1/ACC-α complex constitutes an example of an endogenous ligand for the complex.

The identification of ligands of the complex may be carried out by conventional methods which are well known to the person skilled in the art, such as immunoprecipitation, the two-hybrid method, immunohistochemistry.

In a preferred manner, the method of identification according to the invention comprises:

a) contacting the BRCA1/ACC-α complex with a biological specimen under conditions permitting the formation of interactions between the complex and any ligands;

b) the detection of interactions between the BRCA1/ACC-α complex and the said ligands; and

c) the identification of the ligands interacting with the complex.

The BRCA1/ACC-α complex placed in contact with the biological specimen can be advantageously labelled with the aid of detectable labels such as fluorescent labels or by a radioisotope.

The candidate molecules or ligands of the methods of screening or identification described above include peptide compounds, peptidomimetics or other organic compounds. These molecules can be endogenous, natural or synthetic or may be mixtures of compounds.

The candidate molecules may be structurally defined or not, as is generally the case where extracts are concerned.

“Peptidomimetics” are understood here to mean an organic molecule which mimics certain properties of peptides, for example their binding specificity and/or their physiological activity. Peptidomimetics are generally obtained by modification of the structure of a peptide. These modifications tend in particular to improve the resistance to enzymatic degradation, the bioavailability of the compound or its pharmacokinetic properties.

Within the scope of the present invention, antibodies or aptamers constitute examples of candidate molecules.

Aptamers constitute a class of molecules which represent an alternative to antibodies in terms of molecular recognition. Aptamers are sequences of oligonucleotides having the capacity to recognise virtually any class of target molecules with a high affinity and specificity. Such ligands can be isolated by screening known as SELEX (Systematic Evolution of Ligands by EXponential enrichment) of a bank of random sequences, as described in Tuerk and Gold (1990). The bank of random sequences can be obtained by synthesis of DNA by combinatory chemistry. In such a bank, each member is a linear oligomer, possibly chemically modified, corresponding to a unique sequence. The possible modifications, applications and advantages of this class of molecules have been the subject of a study by Jayasena (1999).

Candidate molecules thus identified, capable of modulating the interaction between BRCA1 and ACC-α or of constituting a ligand of the complex, can be used for the treatment and/or the prevention of cancers of the breast and/or of the ovary, particularly familial forms of cancer of the breast and/or of the ovary.

The invention also relates to a method of in vitro diagnosis of a predisposition to cancer of the breast and/or of the ovary, comprising the determination of a modification of the BRCA1/ACC-α interaction in a subject relative to a control population, the modification of the BRCA1/ACC-α interaction being associated with a variation of the risk of developing a cancer of the breast and/or of the ovary.

“Modification of the BRCA1/ACC-α interaction” is understood to mean in particular the absence or the presence of detectable BRCA1/ACC-α complex in the said subject, or a quantitative or qualitative difference in the BRCA1/ACC-α complex relative to a control population. The presence of the BRCA1/ACC-α complex could for example be detected by a method such as co-immunoprecipitation, for example with the aid of the D16 antibody (Santa Cruz Biotechnology, California, USA) directed against BRCA1, and a polyclonal rabbit antibody directed against the amino-terminal MEDPSPLAQPLELNQ, SEQ ID No. 1) and carboxy-terminal (AEVIRILSTMDSPST, SEQ ID No. 2) ends of the human ACC-α protein.

“Quantitative difference” is understood to mean a detectable variation in the quantity of BRCA1/ACC-α complexes.

“Qualitative difference” is understood to mean in particular a variation likely to modify the biological activity of the complex, such as for example an increased or decreased stability, or a modification of the binding specificity of the complex.

According to another aspect, the invention relates to an anti-human ACC-α antibody directed against the peptides of sequence SEQ ID No. 1 and SEQ ID No. 2 of the human ACC-α protein.

Such an antibody is particularly useful for detecting the presence of a BRCA1/ACC-α complex, for example by immunoprecipitation. The use of such an anti-ACC-α antibody can also permit the cellular localisation of the ACC-α protein as well as the localisation of the ACC-α/BRCA1 complex in the same cells, by an immunochemical technique or by immunofluorescence for example. A diagnostic application may comprise the observation of cells under the microscope in order to detect the existence of this complex and the expected localisation of this complex.

Therefore the present invention also proposes a method of detection ex vivo of a BRCA1/ACC-α complex comprising placing an antibody according to the invention in contact with a biological specimen under conditions permitting the immunoprecipitation of ACC-α and the detection of BRCA1 in the precipitate, the presence of BRCA1 in the precipitate being indicative of the formation of a BRCA1/ACC-α complex.

The present invention also relates to antibodies directed against the BRCA1/ACC-α molecular complex, characterised in that they do not interact with the BRCA1 or ACC-α proteins alone. The selectivity of such antibodies relative to the BRCA1/ACC-α complex can in particular be obtained by producing antibodies which recognise the BRCA1/ACC-α complex, for example by immunisation in the animal, and by carrying out a step of negative selection, that is to say by eliminating the antibodies produced having a reactivity crossed with the isolated BRCA1 protein and the isolated ACC-α protein. Such a negative selection can be carried out by immunoaffinity chromatography.

The antibodies may be poly- or monoclonal antibodies or fragments thereof, chimeric antibodies, particularly humanised or immunoconjugated.

The polyclonal antibodies can be obtained from the serum of an animal immunised against a protein according to the usual modes of operation.

According to an embodiment of the invention, it is possible to use as antigen an appropriate peptide complex, such as a complex associating the minimal domain of BRCA1 and of ACC-α, which can be coupled by means of a reactive residue to a protein (such as patella haemocyanin KLH) or another peptide. Rabbits are immunised with the equivalent of 1 mg of the peptide antigen according to the procedure described by Benoit et al (1982). At intervals of four weeks, the animals are treated with injections of 200 μg of antigen and bled 10 to 14 days later. After the third injection, the antiserum is examined to determine its capacity to bind with the peptide antigen radiomarked with iodine, prepared by the chloramine-T method and is then purified by chromatography on a carboxymethylcellulose (CMC) ion exchange column. The antibody molecules are then collected in the mammals and isolated until the desired concentration is reached by methods which are well known to the person skilled in the art, for example using DEAE Sephadex to obtain the IgG fraction.

In order to increase the specificity of the polyclonal serum, the antibodies can be purified by immunoaffinity chromatography using solid phase immunising polypeptides. The antibody is placed in contact with the solid phase immunising polypeptide for a sufficient time to cause the polypeptide to immunoreact with the antibody molecule in order to form a solid phase immunological complex.

The monoclonal antibodies can be obtained using the conventional method of culture of hybridomes described by Köhler and Milstein (1975).

The antibodies or antibody fragments according to the invention can be for example chimeric antibodies, humanised antibodies, fragments Fab and F(ab′)2. They can also be in the form of labelled antibodies or immunoconjugates.

The antibodies according to the invention, particularly the monoclonal antibodies, can in particular be used for the detection ex vivo by immunochemistry of the BRCA1/ACC-α complex, for example by immunofluorescence, marking with gold, immunoperoxidase, particularly in the screening methods described above.

The following examples illustrate the invention.

### EXPERIMENTAL PROCEDURE

Plasmid Constructions
Eukaryotic Expression Vectors:

The plasmids pUHD 10.3 expressing Brca1 either of the wild type or of the Δ11 form were prepared as described in Bachelier, R. et al (2000). The plasmid pcDNA3β-BRCA1wt expressing the human form of BRCA1 has been previously described (6).
Expression Vectors in Bacteria

The plasmid pGEX-BRCT was obtained by amplification by polymerase chain reaction (PCR) of the nucleotides 4747 to 5436 of Brca1 using the cloned murine Brca1 cDNA as template, and the following primers: 5′-GCGAATTCACATCTTCAGAAGAAAGAGC-3′ (SEQ ID No. 3) and 5′-GCGTCGACTTAATCATTGGAGTCTTGTGG-3′ (SEQ ID No. 4). The polymerase chain reaction product of 0.7 kb was cloned in the sites EcoRI/SalI of pGEX-4T-1 (Amersham Pharmacia Biotech). Shorter constructions with the first or second modules of the tandem BRCT were obtained with the following primers: 5′-GCGAATTCACATCTTCAGAAGAAAGAGC-3′ (SEQ ID No. 5) and 5′-GCGTCGACTCAGCCCTTGAAGAGCTTTTCC-3′ (SEQ ID No. 6) for the construction pGEX-BRCT A, 5′-GCGAATTCCGGGAAAAGCTCTTCAAGG-3′ (SEQ ID No. 7) and 5′-GCGTCGACTTAATCATTGGAGTCTTGTGG-3′ (SEQ ID No. 8) for the construction pGEX-BRCT B. A longer form pGEX-BRCT L containing the nucleotides 4534 to 5436 of Brca1 was also prepared using the following primers: 5′-GCGAATTCGAAGGAACCCCATACCTG-3′ (SEQ ID No. 9) and 5′-GCGTCGACTTAATCATTGGAGTCTTGTGG-3′ (SEQ ID No. 10). A mutant pGEX-BRCT (M) was produced with the same primers as those used for wild-type GST-BRCT using a Brca1 cDNA having the non-sense mutation 1777X as template. The plasmid pGEX-BRCTh expressing the human form of the BRCT tandem was obtained by amplification of the nucleotides 4917 to 5592 using cDNA of BRCA1 inserted into pCDNA3β and the following primers: 5′-GCGGATCCACAGCTTCAACAGAAAGGG-3′ (SEQ ID No. 11) and 5′-GCGTCGACTCAGTAGTGGCTGTGGGG-3′ (SEQ ID No. 12). Mutants pGEX-BRCTh were produced with the same primers as those used for wild-type GST-BRCTh using a BRCA1 cDNA having the non-sense mutation R1835X or Y1853X, or the false-sense mutations A1708E, P1749R, or M1775R, as template.

Cellular Culture

The cells were cultivated in a DMEM medium with 10% foetal calf serum added. The MCF7 cells were cultivated in the same medium with 5 mg/ml of insulin.

For the radiolabelling, the cells were cultivated for an hour in a DMEM medium without methionine, then for three hours in a DMEM medium without methionine which contains 250 μCi of methionine labelled with [³⁵S].

For the transfection, the standard method of precipitation with calcium phosphate was used (Gibco BRL) with 5 μg of total plasmid DNA. Cells were washed 48 hours after transfection.

Co-Sedimentation Tests with GST:

The proteins for fusion with GST were synthesised in Escherichia coli, strain JM 109 (Promega) transformed with pGEX-4T-1, pGEX-BRCT, pGEX-BRCT A, pGEX-BRCT B, pGEX-BRCT L, pGEX-BRCT M or pGEX-BRCTh as well as the mutated forms of BRCTh (A1708E, P1749R, M1775R, R1835X and Y1853X). They were purified by affinity chromatography on Glutathione-Sepharose. The test of binding by co-sedimentation was carried out in vitro by incubating cellular lysates in the presence of non-recombinant GST proteins or GST fusion proteins and Glutathione-Sepharose beads. 10 μg of GST or fusion GST were used in each binding test. The protein complexes were carefully washed (50 mM Tris HCl pH 7.6, 100 mM KCl, 0.05% Tween 20, 1 mM DTT, 0.2 mM PMSF) to eliminate the non-specific protein interactions and released by heating at 100° C. in loading buffer 5× SDS-PAGE and 100 mM DTT. The proteins were analysed by SDS-PAGE and visualised by autoradiography, staining with silver (Bio-Rad SilverStain) or staining with Coomassie blue according to the nature of the lysate used

Preparation of the Total Cellular Extracts:

The cells were lysed in a gentle manner by thermal shock (nitrogen/37° C.) in the following buffer: 25 mM Tris HCl pH 7.6, 1 mM EDTA, 1 mM EGTA, 0.2 mM PMSF, 10 μg/ml of leupeptin, pepstatin A and aprotinin.

Immunoprecipitation and Western Blot Analysis:

The immunoprecipitations of endogenous proteins or proteins overexpressed in vivo were carried out in a lysis buffer with 1 μg of specific antibody (anti-BRCA1 OP92, Oncogene Research Products; anti-Brca1 D16, Santa Cruz Biotechnology; anti-ACC-α L3J74) and the protein G-sepharose for two hours at 4° C. After precipitation the beads were washed three times with the lysis buffer and the proteins were eluted by heating for five minutes in the SDS loading buffer. The proteins were separated on 6 to 10% polyacrylamide gels containing SDS (SDS-PAGE) and loaded onto membranes of poly(vinylidene difluoride) (PVDF) (Immobilon-P, Millipore). The membranes were saturated in TBS containing 0.05% of Tween 20 and 5% of milk powder. The incubations with the primary and secondary antibodies were carried out in TBS containing 0.05% of Tween 20 and 2% of milk powder, and the proteins were detected by chemiluminescence (ECL, Amersham Pharmacia Biotech or Super Signal West Dura Extended, Pierce).

Sub-Cellular Fractionation:

The cytosolic fraction was obtained after incubation of the cells for 10 minutes on ice in a RYMO buffer. (10 mM Tris HCl pH 7.6, 1 mM MgCl₂, 0.5 mM of CaCl₂, 0.25M saccharose, 0.6% NP40) and centrifugation (2000 rpm, 5 minutes). The nuclear pellet was then clarified for 10 minutes on ice in a RIPA buffer (137 mM NaCl, 2.7 mM KCl, 25 mM Tris pH 8, 1% NP40) and centrifuged at 13000 rpm for five minutes to eliminate the insoluble materials. All the fractions were adjusted to 150 mM NaCl, 25 mM Tris pH 7.6 and protease inhibitors were added (2 mM PMSF, 10 μg/ml of leupeptin, pepstatin A and aprotinin). Fractions were tested for their protein concentration by the Bradford method (Bio-Rad Protein Assay) and subjected to Western Blot analyses with antibodies directed against β-tubulin (KMX-1, Roche Molecular Biochemicals) or p300 (N-15, Santa Cruz Biotechnology).

Peptide Sequencing by Mass Spectrometry
Digestion in Gel:

The relevant protein of 210 kDa coloured with Coomassie blue was excised from the SDS-PAGE gel then washed with 50 mM of ammonium acetate buffer, pH 7 for an hour. The supernatant was discarded and the slice of gel washed in a 50/50% (v/v) solution of acetonitrile/25 mM of ammonium acetate, pH 7.5, for an hour and finally with pure water before complete dehydration in a vacuum centrifuge. The pieces of gel were rehydrated with a minimal quantity of a modified porcine trypsin solution (PROMEGA Madison, Wis., USA) containing 25 μg of protease. If necessary, additional buffer was added until the piece of gel was completely rehydrated. Digestion took place at 37° C. for four hours.
Mass Spectrometry: MS-MALDI:

The mass spectra of the tryptic digestion products were obtained on a mass spectrometer MALDI-TOF Voyager Elite X1 (Perspective Biosystems) equipped with delayed extraction. The instrument was used in reflectron mode. 1 μl of the digestion product was deposited directly on the specimen substrate and mixed with 1 μl of a saturated 2,5-dihydrobenzoic acid solution. A list of peptide masses was obtained for each protein digestion product. This peptide mass profile was then processed with appropriate software in order to identify the proteins (MS-FIT:http://prospector.ucsf.edu/ucsfhtml3.2/msfit.htm, or PROFOUND:http://129.85.19.192/prowlcgi/ProFound.ex). The tryptic peptides of the protein of 210 kDa were identified from the mass chart of the tryptic fragments. The tryptic digestion products were extracted twice with a 50/50% (v/v) solution of acetonitrile/25 mM of ammonium acetate pH 7.5. The digestion solution and the extracts were reassembled, dried in a vacuum centrifuge and desalinated with ZipTip C18 (Millipore, Bedford, Mass., USA) before MS/MS analysis.
MS/MS Nanospray:

A Q-TOF instrument (Micromass, Manchester, UK) was used with a Z-Spray ion source functioning in “nanospray” mode. Approximately 3 to 5 μl of each desalted specimen were introduced into a needle (average sampling needle, PROTAN Inc., Odense, Denmark) for carrying out the MS/MS experiments. The capillary voltage was adjusted to an average voltage of 1000 volts and the specimen cone to 50 volts. Glufibrinopeptide was used to calibrate the instrument for the MS/MS mode. The MS/MS spectra were transformed using MaxEnt3 (MassLynx, Micromass Ltd), and the amino acid sequences were analysed with the aid of PepSeq (BioLynx, Micromass Ltd). The amino acid sequences, the fragments of sequences or the ionic peptide fragments which could be determined were used for screening banks of proteins and EST with the aid of appropriate software: Pepfrag (http://prowl1:rockefeller.edu/prowl/pepfragch.html), Scan (http://dna.standford.edu/scan) or BLAST for the searches for homologies (http://www.ncbi.nlm.nih.gov/blast/blast).

Peptide Sequencing by Edman Degradation:

The preparative co-sedimentation test was carried out on the basis of 5.10⁷ cells NIH3T3 and separated by SDS-PAGE followed by staining with amido black. Under these conditions the relevant band contained a sufficient quantity of proteins (25 pmoles corresponding to 5 μg of the protein 210 kDa) to be analysed by peptide sequencing. The slice of polyacrylamide gel corresponding to the 210 kDa band was excised and incubated with a solution of 0.05M Tris HCl pH 8.6/0.03% SDS containing 0.4 μg of endolysine at 35° C. for 18 hours. The endolytic peptides were separated by HPLC on a DEAE-C18 column with a 0.1% acetonitrile/TFA gradient. The sequencing of the isolated peptides was carried out in accordance with the Edman degradation on a Procise sequencer from Applied Biosystems. The amino acid sequences served to screen the protein database to search for homologies: BLAST (http://www.ncbi.nlm.nih.gov/blast/blast), ExPASy (http://www.expasy.org/tools/).

### RESULTS

Co-Sedimentation Tests with GST and Isolation of the Cytoplasmic Protein Interacting with Brca1:

To identify proteins which interact with the tandem formed from the two BRCT modules of Brca1, the inventors carried out a co-sedimentation test with GST with the aid of the cell line of murine fibroblasts NIH3T3. A GST fusion protein containing the amino acids 1583 to 1812 of murine Brca1, called GST-BRCT, was constructed, expressed in Escherichia coli, and purified on glutathione-sepharose beads. Lysates of entire cells were prepared on the basis of 7.10⁶ NIH3T3 cells labelled with ³⁵S methionine. The lysates were pre-purified with the aid of glutathione-sepharose beads. They were then placed in contact with either beads of glutathione alone, or beads of glutathione with GST added, or beads of glutathione with GST-BRCT added. After several washings, the co-sedimentation complexes were separated by SDS-PAGE and visualised by autoradiography. A major band of 210 kDa was visible only in the GST-BRCT specimen.

In order to determine the sub-cellular localisation of the protein interacting with the BRCT tandem, NIH3T3 cells were separated into nuclear and cytoplasmic fractions. A co-sedimentation test carried out on these two fractions was analysed by SDS-PAGE followed by silver-staining. The results showed that the interaction protein was for the most part cytoplasmic. Good fractionation of the cells was controlled with the aid of antibodies directed against nuclear p300 and cytoplasmic β-tubulin.

ACC-α Identified as Being the Protein Interacting with Brca1:

To identify the protein which interacts with the BRCT tandem of Brca1, a preparative co-sedimentation test was carried out on the basis of 5.10⁷ NIH3T3 cells (representing approximately 10 analytical co-sedimentation tests with GST, described above) and separated by SDS-PAGE. Under these conditions, the 210 kDa band could be detected by staining with Coomassie blue. This indicates that this band contains a sufficient quantity of proteins to be analysed by mass spectrometry. Slices of polyacrylamide gel corresponding to the 210 kDa band were excised. The protein was digested enzymatically into several peptides by trypsin, then the mixture was analysed by MALDI-MS using the reflectron mode. The mono-isotopic masses of peptides originating from the proteolytic digestion were compared with the masses of a peptide database, calculated on the basis of the protein database nr of NCBI with mass tolerances of ±0.2 Da and with partially oxidised methionine residues (m/z values listed in profound and sm-fit programs (see experimental procedure): 920.47, 931.52, 938.49, 948.51, 1034.51, 1045.62, 1065.63, 1084.51, 1087.66, 1092.59, 1097.66, 1165.73, 1179.76, 1199.73, 1232.77, 1235.68, 1267.70, 1300.64, 1308.75, 1322.72, 1365.71, 1438.79, 1493.77, 1571.72, 1791.77). The other parameters were as follows: range of variation of the protein mass of 100-300 kDa; cysteines not modified, maximum of three cleavage sites not taken into account. The first protein was acetyl-CoA carboxylase of Rattus norvegicus (EC 6.4.1.2; swiss prot accession number P 11497) with 16 possible peptides out of the 25 peptides listed had a probability close to 1 (9.9e-1) and was clearly distinguished from the second candidate found during the search (protein KIAA 1286 of Homo sapiens; probability of 4.0e-3).

This result was confirmed by ms/ms experiments on m/z ions 634.32, 720.36 and 786.33 corresponding to the ions doubly charged with peptides 1266.7, 1438.79 and 1570.7 Da produced with a Q-TOF. The peptide sequences deducted from the ions b and y were used to screen the BLAST program. The sequences ¹⁴⁰⁹VEVGTEVTDYR¹⁴¹⁹, ¹⁴³⁵EASFEYLQNEGER¹⁴⁴⁷ and the partial sequence of the peptide 1438.79 (⁹⁶DFTVASP¹⁰⁴) showed 100% homology with the acetyl-CoA carboxylase of Rattus norvegicus and confirmed the previous results.

In order to confirm the identity of the protein interacting with BRCT, a microsequencing of the peptide was also carried out. A preparative co-sedimentation test of the GST using 5.10⁷ NIH3T3 cells was carried out to obtain 25 pmoles of protein intended to be digested and sequenced. A slice of polyacrylamide gel containing 5 μg of the relevant band of 210 kDa (approximately 25 pmoles) was excised. The protein was digested enzymatically into peptides by endolysine, then the peptides were separated by chromatography on DEAE-C18 and sequenced. The sequences ²⁹⁸KAAEEVGYP³⁰⁶ and ²²⁶⁷KDLVEWLEK²²⁷⁵ were obtained. As expected, they showed 100% homology with the acetyl CoA carboxylase of Rattus norvegicus (EC 6.4.1.2; swiss prot accession number P 11497).

Two major forms of ACC of animal origin have been described hitherto: ACC-α of 265 kDa and ACC-β of 280 kDa. The 5 peptide sequences obtained here show 100% homology with the ACC-α of the rat. The murine ACC-α gene was not cloned. Nevertheless, the coding sequence of the α gene is well conserved in the different species. Thus, the cDNA sequence of the rat ACC-α (Lopez-Casillas et al, 1988) is highly similar (90%) to the human ACC-α sequence (Abu-Elheiga L. et al, 1995). The site for binding to biotin which is highly conserved and the putative sites for binding to ATP and to coenzyme A are identical in the human, the rat and yeast.

ACC-α Binds to the Tandem of BRCT Modules in the Epithelial Cells of the Mammary Gland:

The inventors have demonstrated that the BRCT tandem of Brca1 binds to endogenous ACC-α in murine fibroblasts Given that the BRCA1 gene is associated with a familial form of breast cancer, the inventors researched the interaction between the BRCT tandem and ACC-α in the mammary gland. The GST co-sedimentation tests were carried out with four murine mammary epithelial cell lines, namely Be4a, I3G2, Tac-2 and J3B1, and the bound proteins were detected with the aid of peroxidase coupled with streptavidin. The capacity of the BRCT tandem to bind ACC-α is conserved in all these cell lines.

A test was carried out to examine whether the BRCT tandem of murine BRCA1 could also be associated with the human form of ACC-α present in the mammary gland. For this purpose they carried out a co-sedimentation test with two cell lines of human mammary epithelium (HBL 100 and MCF 7). A study by Western Blot, with the aid of peroxidase coupled with streptavidin, showed that the capacity of the murine BRCT tandem to bind ACC-α was conserved in the human mammary epithelial cells.

The sequences of the two BRCT domains of murine BRCA1 share 75% and 58% identity respectively with the human BRCA1 protein (Callebaut I. and Mornon J. P., 1997). Furthermore, the cDNA sequence of the rat ACC-α (Lopez-Casillas et al, 1988) is highly similar (90%) to the sequence of human ACC-α (Abu-Elheiga L. et al, 1995). The results represented here indicate that the murine BRCT tandem binds just as well to the human form of ACC-α as to the murine form. Consequently in order to characterise this interaction further the inventors researched the capacity of the human BRCT tandem to bind the murine and human forms of ACC-α. Co-sedimentation tests were carried out on the cell lines of murine fibroblasts NIH3T3 and the human mammary epithelial line HBL 100. The results show that the interaction between the human BRCT tandem and the ACC-α is maintained in the murine and human species.

Together, these data demonstrate a protein-protein interaction which is well conserved between the BRCT tandem of BRCA1 and the ACC-α protein in the murine and human species.

ACC-α Interacts Specifically with the Whole of the Tandem of the BRCT Modules of Brca1:

In order to map the domain of interaction of ACC-α on the BRCT tandem of Brca1, co-sedimentation tests with six distinct fusion proteins were carried out. The protein complexes of co-sedimentation were resolved by SDS-PAGE and visualised by Western Blot. The ACC protein containing the biotin was detected using peroxidase coupled with streptavidin, and as streptavidin has a high affinity for biotin it is therefore a very sensitive probe for detecting ACC-α (Witters, L. A. et al, 1994). The results show that the fragment GST-BRCT L containing the residues 1512-1812 of Brca1, like GST-BRCT, interact with ACC-α. However, the fragment GST-BRCT A which lacks the sequences of the domain B of BRCT is incapable of combining with ACC-α. Likewise, the fragment GST-BRCT B which lacks the sequences of the BRCT A domain does not bind ACC-α. GST-Brca1 N containing the 220 N-terminal residues of Brca1, like GST by itself, was not capable of capturing ACC-α, which demonstrates the specificity of the interaction between ACC-α and the carboxy-terminal region of Brca1.

Numerous familial mutations have been found in the carboxy-terminal segment of BRCA1. One of these, R1835X, localised in the distal domain of BRCT, introduces a non-sense codon which suppresses the last 29 amino acids of Brca1 and thus breaks the second BRCT domain. To determine whether this mutation could abolish the interaction of the BRCT tandem with ACC-α, the mutation W1777X which mimics the human mutation R1835X was introduced on the murine cDNA of Brca1 and a co-sedimentation test was carried out with the mutated tandem (GST-BRCT M). The germinal mutation abolishes the interaction with ACC-α. The effect of this mutation has also been confirmed by introduction of the mutation R1835X into the human cDNA of BRCA1. The corresponding truncated GST-BRCTh fusion protein does not bind ACC-α. The study of other familial mutations such as the mutations A1708E, P1749R or M1775R, which create an amino acid substitution in the BRCT A domain, in the sequence binding the two BRCT domains or in the BRCT B domain respectively, or the non-sense mutation Y1853X which leads to a truncated BRCT B domain, shows that these mutations also abolish the binding of BRCA1 to ACC-α.

All of these data indicate that the minimal BRCA1 sequence required for combining with ACC-α corresponds precisely to the tandem of the BRCT domains, which signifies that the two BRCT units are implicated in the interaction with ACC-α.

BRCA1 and its Short Form Brca1-Δ11 Combine in Vivo with ACC-α:

To determine whether the BRCA1 protein can interact with ACC-α in vivo, co-immunoprecipitation experiments were carried out with lysates of transfected Bosc cells. For this purpose, the Bosc cells were transfected with expression plasmids coding for Brca1 of total length or the short form Brca1-Δ11. The lysates prepared on the basis of these transfected cells were immunoprecipitated with the antibody D16 (Santa Cruz Biotechnology) directed against BRCA1 and each immunoprecipitate was fractionated by SDS-PAGE. The presence of endogenous ACC-α in these immunoprecipitates, was then determined by Western Blot analysis with the aid of peroxidase coupled with streptavidin. The endogenous ACC-α was co-immunoprecipitated from cells expressing Brca1 and from cells expressing the short form Brca1-Δ11. For more advanced study of whether the human form of BRCA1 could combine with endogenous ACC-α, Bosc cells were transfected with a plasmid expressing the BRCA1 protein. Lysates were immunoprecipitated with the antibody OP92 (Oncogene Research Products) directed against BRCA1 and the presence of endogenous ACC-α in the co-immunoprecipitates was analysed by Western Blot with the aid of peroxidase coupled with streptavidin. As expected, human BRCA1 coprecipitates with endogenous ACC-α.

Because BRCA1 is principally a nuclear protein, the results obtained in the co-sedimentation tests and the results concerning the cytoplasmic localisation of ACC-α raise the question of the physiological significance of the interaction between BRCA1 and ACC-α. The inventors re-examined the sub-cellular localisation of BRCA1 and of the murine isoform Brca1-Δ11 expressed temporarily in Bosc cells. For this purpose, fractionation techniques were used to study the sub-cellular localisation of BRCA1 and of the murine form Brca1-Δ11 (Bachelier, R. et al, 2000). After cellular fractionation, nuclear and cytoplasmic extracts of the transfected cells were separated by SDS-PAGE and subjected to Western Blot analysis. As expected, the human endogenous BRCA1 protein was detected principally in the nuclear fraction, whilst BRCA1 expressed in an ectopic manner was principally cytoplasmic. These results are in agreement with the preceding observations (Bachelier, R. et al, 2000). Furthermore, the exogenous murine isoform Brca1-Δ11 was principally present in the cytoplasmic fraction

Taken together, these results indicate that the ACC-α which is localised in the cytoplasm binds the BRCA1 proteins of total length and the short form BRCA1-Δ11 expressed in an ectopic manner, via the tandem of the BRCT domains present both in the human and the murine forms.

No antibody directed against the human form of ACC-α is available on the market, and therefore the inventors prepared in the rabbit a polyclonal antibody directed against the amino-terminal ends (MEDPSPLAQPLELNQ, SEQ ID No. 1) and the carboxy-terminal ends (AEVIRILSTMDSPST, SEQ ID No. 2) of the human ACC-α protein. This antibody was then immunopurified against these two peptide sequences. The specificity of this anti-ACC-α antibody called L3J74 was tested by the Western Blot technique in several cell lines (human lines: HBL 100, MCF7, BT20, Bosc, HCC1937; murine line: NIH3T3) and several lysates of tissue (breast, ovary, pancreas) under experimental conditions making it possible to distinguish clearly the α and β forms of ACC. The results showed that L3J74 was specific to the α form of ACC and that its affinity for the human form was very much greater than that observed for the murine form of the protein. This antibody was then tested by an immunoprecipitation technique on different cellular lysates (HBL100, Bosc, NIH3T3). Two hundred nanograms of this antibody permit the immunoprecipitation of ACC-α in a lysate of 5×10⁶ cells.

In order to show that the ACC-α protein combines with BRCA1 in vivo, under physiological conditions, co-immunoprecipitation experiments were carried out on human mammary gland epithelial cells of cell line HBL 100. The immunoprecipitation of BRCA1 results in the coprecipitation of ACC-α. Conversely, the immunoprecipitation of ACC-α with the antibody L3J74 results in the coprecipitation of BRCA1. These results show clearly that BRCA1 and ACC-α form an endogenous complex in vivo.

### BIBLIOGRAPHY

Abu-Elheiga L. et al. (1995). Human acetyl-CoA carboxylase: characterization, molecular cloning, and evidence for two isoforms. Proc. Natl. Sci. USA. 92: 4011-4015.
Anderson S. F. et al. (1998). BRCA1 protein is linked to the RNA polymerase II holoenzyme complex via RNA helicase A. Nat. Genet. 19: 254-256.
Bachelier, R. et al. (2000) Int J Cancer, 88, 519-524.
Bennett L. M. et al (1995) Genomics, 29, 576-581.
Benoit et al., (1982) PNAS USA, 79, 917-921.
Bork P. et al. (1997). A superfamily of conserved domains in DNA damage-responsive cell cycle checkpoint proteins. The FASEB J. 11: 68-76.
Callebaut I. & Mornon, J. P. (1997). From BRCA1 to RAP1: a widespread BRCT module closely associated with DNA repair. FEBS. 400: 25-30.
Cantor S. B. et al (2001) Cell, 105, 149-160.
Chapman M. S. et Verma I. M. (1996). Transcriptional activation by BRCA1, Nature 382: 678-679.
Chen J. et al. (1988). Stable interaction between the products of the BRCA1 and BRCA2 tumor suppressor genes in mitotic and meiotic cells. Mol. Cell. 2: 317-328.
Critchlow S. E. et al (1997). Mammalian DNA double-strand break repair protein XRCC4 interacts with DNA ligase IV. Current Biol. 7: 588-598.
Ford D. et al. (1998). Genetic heterogeneity and penetrance analysis of the BRCA1 BRCA2 genes in breast cancer families. Am. J. Hun. Genet. 62:676-688.
Hakem R. et al (1995). The tumor suppressor gene BRCA1 is required for embryonic cellular proliferation in the mouse, Cell. 85: 1009-1023.
Hakem R. et al. (1997). The tumor suppressor gene BRCA1 is required for embryonic cellular proliferation in the mouse. Cell. 85: 1009-1023.
Jayasena (1999) Aptamers an emerging class of molecules that rival antibodies in diagnostics. Clin. Chem. 45(9):1628-50.
Köhler et Milstein (1975) Nature, 256, 495-497.
Koonin E. V. et al. (1996). BRCA1 protein products Functional motifs. Nat. Genet. 13:287-289.
Larson J. S. et al. (1997). A BRCA1 mutant alters G₂-M cell cycle control in human mammary epithelial cells, Cancer Res. 57: 3351-3355.
Lopez-Casillas, F., Bai, D. H., Luo, X., Kong, I. S., Hermodson, M. A. & Kim, K. H. (1988) Proc. Natl. Acad. Sci. USA 85, 5784-5788.
Magdinier F. et al. (1999). BRCA1 expression during prenatal development of the human mammary gland. Oncogene. 18: 4039-4043.
Masson M. et al. (1998). XRCC1 is specifically associated with poly (ADP-Ribose) polymerase and negatively regulates its activity following DNA damage. Mol. Cell. Biol. 18: 3563-3571.
Miki Y. et al. (1994). A strong candidate for the breast and ovarian cancer susceptibility gene BRCA1, Science. 286: 66-71.
Monteiro A. N. A et al. (1996). Evidence for a transcriptional activation function of BRCA1 C-terminal region. Proc. Natl. Acad. Sci. USA. 93: 13595-13599.

Nash R. et al. (1997). XRCC1 protein interacts with one of two distinct forms of DNA ligase III, Biochemistry, 36: 5207-5211.
Ouchi T. et al. (1998). BRCA1 regulates p53-dependent gene expression. Proc. Natl. Acad. Sci. USA. 95: 2302-2306.
Scully R. et al. (1997a). Association of BRCA1 with Rad51 in mitotic and meiotic cells. Cell. 88: 265-275.
Scully R. et al. (1997b). BRCA1 is a component of the RNA polymerase II holoenzyme. Proc. Natl. Acad. Sci. USA. 94: 5605-5610.
Scully R. et al. (1998o). Dynamic changes of BRCA1 subnuclear location and phosphorylation state are initiated by DNA damage. Cell. 90: 425-435.
Smith A. A. et al. (1992). Alleles losses in the region 17q12-21 in familial breast and ovarian cancer involve the wild-type chromosome. Net. Genet. 2: 128-131.
Smith T. M. et al. (1998). Complete genomic sequence and analysis of 117 kb of human DNA containing the gene BRCA1. Genome Research. 6: 1029-1049.
Tuerk et Gold (1990) Systematic evolution of ligands by exponential enrichment. RNA ligands to bacteriophage T4 DNA polymerase. Science, 3:249(4968):505-10.
Wang Q. et al. (1998). BRCA1 binds c-Myc and inhibits its transcriptional and transforming activity in cells. Oncogene. 17: 1939-1948.
Witters, L. A., Widmer, J., King, A. N., Fassihi, K. & Kuhajda, F. (1994) Int. J. Biochem. 26, 589-594.
Wooster R. et al. (1995). Identification of the breast cancer susceptibility gene BRCA2, Nature, 378: 789-792.
Wu L. C. et al. (1996). Identification of a RING protein that can interact in vivo with the BRCA1 gene product. Nat. Genet. 14: 430-440.
Yarden R. et al. (1999). BRCA1 interacts with components of the histone deacetylase complex. Proc. Natl. Acad. Sci. USA. 96: 4983-4988.
Yu X. et al. (1998). The C-terminal (BRCT) domains of BRCA1 interact in vivo with CHP, a protein implicated in the CLBP pathway of transcriptional repression. J. Biol. Chem. 273: 25388-25392.
Zhang H. et al. (1998a). BRCA1 physically associates with p53 and stimulates its transcriptional activity. Oncogene. 16: 1713-1721.
Zhang X. et al. (1998b). Structure of an XRCC1 BRCT domain: a new protein-protein interaction module. The EMBO J. 17: 6404-6411.

1-10. (canceled)

11. A molecular complex comprising:
a first polypeptide comprising a sequence of amino acids 1640 to 1863 of human BRCA1 protein or the same sequence of amino acids of the BRCA1 protein in another species of animal, and
a second polypeptide comprising a fragment of ACC-α protein capable of binding the said first polypeptide.

12. A method of screening in vitro of molecules useful for prevention or treatment of cancer of breast and/or of ovary in which molecules are tested for their capacity to modulate interaction between BRCA1 and ACC-α proteins.

13. The method of screening according to claim 12, comprising:
a) contacting, in any order whatsoever, two different partners or three different partners selected from the group consisting of a first polypeptide comprising the sequence of amino acids 1640 to 1863 of the human BRCA1 protein or the same sequence of amino acids of the BRCA1 protein, a second polypeptide comprising a fragment of the ACC-α protein capable of binding the said first polypeptide, and a candidate molecule;
b) incubating said partners for a sufficient time to permit any interaction thereof;
c) in the case where only two different partners have been selected at step a), adding the third partner selected from the said group and incubating for a sufficient time to permit any interaction thereof;
d) determining the capacity of the candidate molecule to modulate the interaction between BRCA1 and ACC-α, wherein said capacity is indicative of a molecule useful for the prevention or the treatment of cancer of the breast and/or of the ovary.

14. The method of screening according to claim 12, comprising:
a) contacting a first polypeptide comprising the sequence of amino acids 1640 to 1863 of the human BRCA1 protein or the same sequence of amino acids of the BRCA1 protein with a second polypeptide comprising a fragment of the ACC-α protein capable of binding the said first polypeptide, one at least of the polypeptides being labelled in a detectable manner;
b) adding a candidate molecule capable of modulating the interaction;
c) incubating the said polypeptides in the presence of the candidate molecule under conditions and for a period of time which are sufficient for the binding between the said polypeptides to take place;
d) quantifying the number of labelled molecules bound in the presence of increasing concentrations of the candidate molecule, wherein a modification of the number of labelled molecules bound with increasing concentrations of the candidate molecule is indicative of a molecule useful for the prevention or the treatment of cancer of the breast and/or of the ovary.

15. The method of screening according to claim 12, wherein said molecule capable of modulating the BRCA1/ACC-α interaction is an agonist.

16. The method of screening according to claim 12, wherein the said molecule capable of modulating the BRCA1/ACC-α interaction is an antagonist.

17. A method of identification ex vivo of molecules constituting endogenous ligands of a BRCA1/ACC-α complex comprising:
a) contacting a BRCA1/ACC-α complex with a biological specimen under conditions permitting the formation of interactions between the complex and any ligands;
b) detecting interactions between the BRCA1/ACC-α complex and the said ligands; and
c) identifying ligands interacting with the complex as molecules constituting endogenous ligands of the BRCA1/ACC-α complex.

18. A method of in vitro diagnosis of a predisposition to cancer of the breast and/or of the ovary, comprising determining a modification of a BRCA1/ACC-α interaction in a subject relative to a control population, the modification of the BRCA1/ACC-α interaction being associated with a variation of the risk of developing a cancer of the breast and/or of the ovary.

19. An anti-human ACC-α antibody directed against peptides of sequence SEQ ID No. 1 and SEQ ID No. 2.

20. An antibody directed against a BRCA1/ACC-α molecular complex, which does not interact with the BRCA1 or ACC-α proteins alone.
